SEQUENCE LISTING (24 sequences)

SEQ ID NO: 1 - protein, 7 aa, Artificial Sequence
  Synthetic
VVPRSFR

SEQ ID NO: 2 - protein, 7 aa, Artificial Sequence
  Synthetic
VLPRSFR

SEQ ID NO: 3 - protein, 7 aa, Artificial Sequence
  Synthetic
VTPRSFR

SEQ ID NO: 4 - protein, 7 aa, Artificial Sequence
  Synthetic
LLPRSFR

SEQ ID NO: 5 - protein, 7 aa, Artificial Sequence
  Synthetic
LTPRSFR

SEQ ID NO: 6 - protein, 7 aa, Artificial Sequence
  Synthetic
VVPRSYR

SEQ ID NO: 7 - protein, 7 aa, Artificial Sequence
  Synthetic
VLPRSYR

SEQ ID NO: 8 - protein, 7 aa, Artificial Sequence
  Synthetic
VTPRSYR

SEQ ID NO: 9 - protein, 7 aa, Artificial Sequence
  Synthetic
LLPRSYR

SEQ ID NO: 10 - protein, 7 aa, Artificial Sequence
  Synthetic
LTPRSYR

SEQ ID NO: 11 - protein, 7 aa, Artificial Sequence
  Synthetic
LTPRGVN

SEQ ID NO: 12 - protein, 50 aa, Homo sapiens
MSETSRTAFGGRRAVPPNNSNAAEDDLPTVELQGVVPRGVNLQEFLNVTS

SEQ ID NO: 13 - protein, 50 aa, Artificial Sequence
  Synthetic
MSETSRTAFGGRRAVPPNNSNAAEDDLPTVELQGLTPRGVNLQEFLNVTS

SEQ ID NO: 14 - protein, 50 aa, Artificial Sequence
  Synthetic
MSETSRTAFGGRRAVPPNNSNAAEDDLPTVELQGLTPRSYRLQEFLNVTS

SEQ ID NO: 15 - protein, 50 aa, Artificial Sequence
  Synthetic
MSETSRTAFGGRRAVPPNNSNAAEDDLPTVELQGLTPRSFRLQEFLNVTS

SEQ ID NO: 16 - DNA, 35 bp, Artificial Sequence
  Synthetic
gaccttgtgaattcaaaaatgtcagaaacttccag

SEQ ID NO: 17 - DNA, 22 bp, Artificial Sequence
  Synthetic
gtttagaattcacgttcccatc

SEQ ID NO: 18 - DNA, 22 bp, Artificial Sequence
  Synthetic
gatgggaacgtgaattctaaac

SEQ ID NO: 19 - DNA, 23 bp, Artificial Sequence
  Synthetic
ccttcttgaactctgccttcggg

SEQ ID NO: 20 - DNA, 23 bp, Artificial Sequence
  Synthetic
cccgaaggcagagttcaagaagg

SEQ ID NO: 21 - DNA, 45 bp, Artificial Sequence
  Synthetic
tctcagcttcctctagattcacatggaaggtcgtctttgaatctg

SEQ ID NO: 22 - DNA, 30 bp, Artificial Sequence
  Synthetic
gttgacgccccggggcaccaagccctgaag

SEQ ID NO: 23 - DNA, 19 bp, Artificial Sequence
  Synthetic
cttgtcaaattgaattttc

SEQ ID NO: 24 - DNA, 34 bp, Artificial Sequence
  Synthetic
gttgacgccccggggagtcaagccctgaagctcc

(19) United States
(12) Patent Application Publication
(10) Pub. No.: US 2009/0130086 A1
(43) Pub. Date: May 21, 2009

(54) FXIII Variants with Improved Properties

(75) Inventors: Rasmus Roejkjaer, Princeton, NJ (US); Asser Sloth Andersen, Herlev (DK); Marianne Kjalke, Frederikssund (DK); Ole Hvilsted Olsen, Broenshoej (DK); Henning Ralf Stennicke, Kokkedal (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk Health Care AG, Zurich (CH)

(21) Appl. No.: 11/817,039

(22) PCT Filed: Feb. 27, 2006
(86) PCT No.: PCT/EP06/60297

§ 371 Date: Sep. 11, 2008

(30) Foreign Application Priority Data
Feb. 28, 2005 (DK) PA 2005 00296

(51) Int. Cl. A61K 38/48; C12N 9/48

(52) U.S. Cl. 424/94.64; 435/212

(57) ABSTRACT

The present invention concerns variant factor XIII, wherein the rate of activation of said variant by thrombin is faster than for wild type FXIII. Methods for enhancing fibrin clot formation, pharmaceutical compositions and the use for the manufacture of medicaments wherein the variant factor XIII is applied are disclosed.

### FIELD OF THE INVENTION

A method for enhancing fibrin clot formation in a subject and a pharmaceutical composition comprising a variant factor XIII.

### BACKGROUND OF THE INVENTION

Haemostasis is initiated by the formation of a complex between tissue factor (TF) being exposed to the circulating blood following an injury to the vessel wall, and FVIIa which is present in the circulation in an amount corresponding to about 1% of the total FVII protein mass. This complex is anchored to the TF-bearing cell and activates FX into FXa and FIX into FIXa on the cell surface. FXa activates prothrombin to thrombin, which activates FVIII, FV, FXI and FXIII. Furthermore, the limited amount of thrombin formed in this initial step of haemostasis also activates the platelets. Following the action of thrombin on the platelets these—among multiple other events—change shape and expose negatively charged phospholipids on their surface. This activated platelet surface forms the template for the further FX activation and the full thrombin generation. The further FX activation on the activated platelet surface occurs via a FIXa-FVIIIa complex formed on the surface of the activated platelet, and FXa in complex with FVa then converts prothrombin into thrombin. Thrombin then converts fibrinogen into fibrin, which stabilizes the initial platelet plug. FXIIIa finally adheres to the fibrin monomer and cross-link the fibrin fibres and covalently attaches fibrinolysis inhibitors, i.e. alpha2-antiplasmin to the clot, thereby providing mechanical strength and resistance against fibrinolysis.

FVII exists in plasma mainly as a single-chain zymogen, which is cleaved by FXa into its two-chain, activated form, FVIIa. Recombinant activated factor VIIa (rFVIIa) has been developed as a pro-haemostatic agent. The administration of rFVIIa offers a rapid and highly effective pro-haemostatic response in haemophilic subjects with bleedings who can not be treated with coagulation factor products due to antibody formation. Also bleeding subjects with a factor VII deficiency or subjects having a normal coagulation system but experiencing excessive bleeding can be treated successfully with rFVIIa

Pharmacological doses of rFVIIa increases the formation of thrombin on the activated platelet surface. This occurs in haemophiliac subjects lacking FIX or FVIII and therefore missing the most potent pathway for full thrombin formation. Also in the presence of a lowered number of platelets or platelets with a defect function, extra rFVIIa increases the thrombin formation.

FXIII is a transglutaminase. When activated by thrombin to FXIIIa, the enzyme catalyses formation of intermolecular gamma-glutamyl-epsilon-lysine cross-links between fibrin monomers and other substrates. Thereby cross-linked fibrin is formed providing mechanical resistance to the clot. Furthermore a number of antifibrinolytic, prohaemostatic and adhesive proteins are cross-linked to the clot thereby providing a strong fibrin structure with increased mechanical resistance to the dissolution by plasmin and other proteolytic enzymes. Factor XIII is also known as “fibrinoligase” and “fibrin stabilizing factor”. FXIII is found in plasma and in platelets. The enzyme exists in plasma as a tetrameric zymogen consisting of two a-subunits and two b-subunits (designated a₂b₂) and in platelets as a zymogen consisting of two a-subunits (designated a₂).

Both zymogens a₂b₂ and a₂ are activated by thrombin and Calcium. Calcium is being released from the platelets on the aggregation at the site of injury. Thrombin cleaves the peptide bond between amino acid residue 37 and 38 of a-subunits. In case of the a₂b₂-zymogen, the b-subunits are then dissociated from the activated a-subunits. Following the thrombin and calcium activation the active site cysteine on the a-subunit is exposed and the fully activated enzyme is formed. Subjects with severe thrombocytopenia have been found to have low plasma levels of FXIII. Furthermore patients with graft-versus-host complications after allogenic stem cell transplantation and patients undergoing cardiac surgery have decreased plasma levels of FXIII.

It is well known that subjects who bleed excessively in association with surgery or major trauma and need blood transfusions develop more complications than those who do not experience any bleeding. However, also moderate bleedings requiring the administration of human blood or blood products (platelets, leukocytes, plasma-derived concentrates for the treatment of coagulation defects, etc.) may lead to complications associated with the risk of transferring human viruses (hepatitis, HIV, parvovirus, and other, by now unknown viruses). Extensive bleedings requiring massive blood transfusions may lead to the development of multiple organ failure including impaired lung and kidney function. Once a subject has developed these serious complications a cascade of events involving a number of cytokines and inflammatory reactions is started making any treatment extremely difficult and unfortunately often unsuccessful. Therefore a major goal in surgery as well as in the treatment of major tissue damage is to avoid or minimise the bleeding.

To avoid or minimise such bleeding it is of importance to ensure the formation of stable and solid haemostatic plugs that are not easily dissolved by fibrinolytic enzymes. Furthermore, it is of importance to ensure quick and effective formation of such plugs or clots.

Japanese patent application No. 2-167234 A concerns an adhesive for bio-tissue characterized by containing fibrinogen, prothrombin, blood coagulation factor VII, blood coagulation factor IX, blood coagulation factor X, blood coagulation factor XIII, antithrombin, a proteinase inhibitor, and calcium ion.

Japanese patent application No. 59-116213A concerns an aerosol composition for use as tissue glue containing a blood coagulant as an active component. The blood coagulant may be selected from blood coagulation factors I, II, III, IV, V, VII, VIII, IX, X, XI, XII, and XIII, prekallikrein, high polymer kininogen and thrombin. A combination of F XIII and thrombin is preferred.

WO 93/12813 (ZymoGenetics) concerns the use of FXIII for reducing perioperative blood loss in a subject undergoing surgery. The composition may also comprise aprotinin. The FXIII is administered to the subject as a bolus injection, typically one day prior to surgery.

European Patent No. 225.160 (Novo Nordisk) concerns compositions of FVIIa and methods for the treatment of bleeding disorders not caused by clotting factor defects or clotting factor inhibitors.

European Patent No. 82.182 (Baxter Travenol Lab.) concerns a composition of factor VIIa for use in counteracting deficiencies of blood clotting factors or the effects of inhibitors to blood clotting factors in a subject.

International Patent Publication No. WO 93/06855 (Novo Nordisk) concerns the topical application of FVIIa.

Kjalke et al, Thromb Haemost, 1999 (Suppl), 0951 concerns the administration of extra exogenous FVIIa and the effect on the formation of thrombin on the activated platelet surface in a model system mimicking hemophilia A or B conditions.

Activation of FXIII variants have been studied in Isetti and Maurer (2004) Biochemistry, 43:4150-4159; Lee et al. (2002) Exp Mol Med 34:385-390; Trumbo and Maurer (2002) Biochemistry, 41:2859-2868; Ariēns et al. (2000) Blood 96: 988-995; Schroeder et al., (2001), Thromb Res 104: 467-474.

The amino acid sequence LTPRSFR at P₄-P′₃ positions, has been described as the optimal consensus sequence for thrombin cleavage sites (Harris J L et al. Proc Natl Acad Sci USA 2000; 97: 7754-7759, Bianchini E P et al. J Biol Chem. 2002, 277: 20527-20534). The P nomenclature system (. . . P₃, P₂, P₁, P′₁ , P′₂, P′₃ . . . ) is used to assign the individual amino acid positions on the substrate peptides. Hence, for residues of the substrates numbered from P₄ to P′₃, P₄ and P′₃ refer to 4 and 3 residues remote from the cleavage site on the N- and C-terminal side, respectively. The P₁-P′₁ peptide bond becomes hydrolyzed by the enzyme. The corresponding subsites on the enzyme are numbered from S₄ to S′₃ (Schechter and Berger, Biochem Biophys Res Comm 1968, 32:898-902; and 1967, 27:157-162).

There remains a need in the art for an improved, reliable and widely applicable method of enhancing coagulation, quickly forming stable haemostatic plugs and achieving full haemostasis in subjects, in particular in subjects having an impaired thrombin generation.

### SUMMARY OF THE INVENTION

The FXIII variants of the invention can be applied in a method and a pharmaceutical composition which provides quickly forming stable haemostatic plugs when used for treating bleeding episodes in a subject. The FXIII variants of the invention provides a faster (compared to wild type FXIII) activation by thrombin and a faster intermolecular polymerization of fibrin through formation of epsilon-lysine -gamma-glutamyl cross-links during blood clotting.

A first aspect of the invention relates to a method for enhancing fibrin clot formation in a subject comprising the steps:
a) providing a variant factor XIII, wherein the rate of activation of said variant by thrombin is faster than for wild type FXIII;
b) administering an effective amount of the variant factor XIII to a subject.

A further aspect of the invention relates to a pharmaceutical composition comprising a variant factor XIII, wherein the rate of activation of said variant by thrombin is faster than for wild type FXIII.

A further aspect of the invention relates to use of a factor XIII-variant, wherein the rate of activation of said variant by thrombin is faster than for wild type FXIII, for the manufacture of a medicament for treating bleeding episodes in a subject.

In a preferred embodiment of the invention at least one amino acid in a region comprising position 28-41 of the activation peptide has been modified in the factor XIII variant.

In a further preferred embodiment the modification or modifications is selected from the group consisting of T28D, V29F, E30L, L31A, Q32E, V34(G, L), V35(M,K,Q,R,E,H,L,T,N,S,A), P36(L,V), G38(S,A), V39(F,Y,W,R,M), N40(R,K,W,H,Q,A,S) and L41V.

In a further preferred embodiment the modification comprises replacement in position 34-40 by an amino acid sequence selected from the group of VVPRSFR, VLPRSFR, VTPRSFR, LLPRSFR, LTPRSFR, VVPRSYR, VLPRSYR, VTPRSYR, LLPRSYR, LTPRSYR or LTPRGVN.

In a further preferred embodiment of the invention the administration of variant factor XIII is combined with administration of a factor VIIa.

In a further embodiment of the invention both the factor XIII variant and the factor VIIa or factor VIIa variant is human.

In a further embodiment of the invention the factors XIII and VIIa are recombinant human factor XIII and factor VIIa.

In a further preferred embodiment the pharmaceutical composition of the invention is in the form of a kit-of-parts comprising the factor XIII variant and the factor VIIa or variant factor VIIa in separate container means.

A further aspect of the invention relates to a factor XIII variant, wherein at least one amino acid in a region comprising position 28-41 of the activation peptide has been modified, the modification or modifications is being selected from the group consisting of T28D, V29F, E30L, L31A, Q32E, V34(G), V35(M,K,Q,R,E,H,T,N,S,A), P36(L,V), G38(S,A), V39(F,Y,W,R,M), N40(R,K,W,H,Q,A,S) and L41V.

In a preferred embodiment of the invention the residues in position 34-40 of the variant factor XIII has been replaced by the amino acid sequence VVPRSFR or VLPRSFR or VTPRSFR or LLPRSFR or LTPRSFR or VVPRSYR or VLPRSYR or VTPRSYR or LLPRSYR or LTPRSYR or LTPRGVN.

### DESCRIPTION OF THE INVENTION

### Faster Activation of FXIII by Thrombin

The N-terminal 50 amino acid residues of FXIII wildtype has the following sequence:

MSETSRTAFG GRRAVPPNNS NAAEDDLPTV ELQGVVPRGV
NLQEFLNVTS

In a preferred embodiment according to the present invention the wildtype sequence of the factor XIII of the method and composition has been modified by exchanging amino acids close to the thrombin cleavage site resulting in the following N-terminal sequences (modifications in bold)

FXIII(V34L, V35T):
MSETSRTAFG GRRAVPPNNS NAAEDDLPTV ELQGLTPRGV
NLQEFLNVTS
FXIII LTPRSYR:
MSETSRTAFG GRRAVPPNNS NAAEDDLPTV ELQGLTPRSY
RLQEFLNVTS
FXIII LTPRSFR:
MSETSRTAFG GRRAVPPNNS NAAEDDLPTV ELQGLTPRSF
RLQEFLNVTS

The invention is not restricted to methods or compositions wherein the factor XIII variant comprises these sequences but also to methods and compositions wherein other factor XIII variants with increased rate of thrombin activation is used.

In the present context the three-letter or one-letter indications of the amino acids have been used in their conventional meaning as indicated in table 1. Unless indicated explicitly, the amino acids mentioned herein are L-amino acids. It is to be understood, that the first letter in, for example, V34 represent the amino acid naturally present at the indicated position wild-type factor XIII, and that, for example, [V34L]-FXIII designates the FXIII-variant wherein the amino acid represented by the one-lefter code V naturally present in the indicated position is replaced by the amino acid represented by the one-letter code L.

TABLE 1
Abbreviations for amino acids:

| | Tree-letter | |
|---|---|---|
| Amino acid | code | One-letter code |
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |

The term “factor VIIa” or “FVIIa” may be used interchangeably. The term factor VIIa includes zymogen factor VII (single-chain factor VII). The term “factor XIII” or “FXIII” may be used interchangeably.

It will be apparent to those skilled in the art that substitutions can be made outside the regions critical to the function of the factor XIII-molecule and still result in an active polypeptide. Amino acid residues essential to the activity of the factor XIII-polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site-directed mutagenesis using any of the methods known in the art. Particularly useful is the procedure that utilizes a super coiled, double stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with Dpnl, which is specific for methylated and hemimethylated DNA to digest the parental DNA template and to select for mutation-containing synthesized DNA. Other procedures known in the art for creating, identifying and isolating variants may also be used, such as, for example, gene shuffling or phage display techniques.

Within the present invention an “effective amount” of a factor VIIa and an “effective amount” of a factor XIII is defined as the amount of a factor VIIa and a factor XIII sufficient to prevent or reduce bleeding or blood loss, so as to cure, alleviate or partially arrest the disease and its complications.

The amount of a factor VIIa and the amount of a factor XIII administered according to the present invention preferably vary from a ratio of about 1:100 to about 100:1 (μg factor VIIa:μg factor XIII), such as for example about 1:60 to about 25:1, e.g. 1:30 to about 10:1, for example about 1:15 to about 5:1, preferably about 1:7 to about 1:1.

In this context, “subjects with an impaired thrombin generation” means subjects who cannot generate a full thrombin burst on the activated platelet surface and includes subjects less capable of thrombin generation than subjects having a fully functioning, normal haemostatic system, including a normal amount and function of coagulation factors, platelets and fibrinogen, and includes subjects lacking FIX and/or FVIII (haemophilia A and B) or having defective FIX and/or FVIII or having inhibitors against FIX and/or FVIII; subjects lacking FXI; subjects with a lowered number of platelets or platelets with a defective function (e.g., thrombocytopenia or thrombasthenia Glanzmann or subjects with excessive bleeds); and subjects having lowered levels of prothrombin, FX or FVII.

Subjects with lowered plasma concentrations of fibrinogen (e.g., multitransfused subjects as a consequence of multiple trauma or extensive surgery) do also suffer from the formation of looser and unstable fibrin plugs which are easily dissolved.

The term “full haemostasis” means the formation of a stable and solid fibrin clot or plug at the site of injury which effectively stops the bleeding and which is not readily dissolved by the fibrinolytic system.

The term “activity of factor VIIa” or “factor VIIa-activity” means the ability to generate thrombin; the term also includes the ability to generate thrombin on the surface of activated platelets in the absence of tissue factor.

The term “enhancement of the normal haemostatic system” means an enhancement of the ability to generate thrombin.

As used herein the term “bleeding disorder” reflects any defect, congenital, acquired or induced, of cellular or molecular origin that is manifested in bleedings. Examples are clotting factor deficiencies (e.g. haemophilia A and B or deficiency of coagulation factors XI or VII), clotting factor inhibitors, defective platelet function, thrombocytopenia or von Willebrand's disease.

The term “bleeding episodes” is meant to include uncontrolled and excessive bleeding which is a major problem both in connection with surgery and other forms of tissue damage. Uncontrolled and excessive bleeding may occur in subjects having a basically normal coagulation system (these subjects do however develop a coagulopathy as a result of the bleeding—dilution of coagulation proteins, increased fibrinolysis and lowered platelets due to a dilution effect of the bleeding) and subjects having coagulation or bleeding disorders. Clotting factor deficiencies (haemophilia A and B, deficiency of coagulation factors XI or VIII) or clotting factor inhibitors may be the cause of bleeding disorders. Excessive bleedings also occur in subjects with a normally functioning blood clotting cascade (no clotting factor deficiencies or -inhibitors against any of the coagulation factors) and may be caused by a defective platelet function, thrombocytopenia or von Willebrand's disease. In such cases, the bleedings may be likened to those bleedings caused by haemophilia because the haemostatic system, as in haemophilia, lacks or has abnormal essential clotting “compounds” (such as platelets or von Willebrand factor protein) that causes major bleedings. In subjects who experience extensive tissue damage in association with surgery or vast trauma, the normal haemostatic mechanism may be overwhelmed by the demand of immediate haemostasis and they may develop bleeding in spite of a basically (pre-trauma) normal haemostatic mechanism. Achieving satisfactory haemostasis also is a problem when bleedings occur in organs such as the brain, inner ear region and eyes with limited possibility for surgical haemostasis. The same problem may arise in the process of taking biopsies from various organs (liver, lung, tumour tissue, gastrointestinal tract) as well as in laparoscopic surgery. Common for all these situations is the difficulty to provide haemostasis by surgical techniques (sutures, clips, etc.) which also is the case when bleeding is diffuse (haemorrhagic gastritis and profuse uterine bleeding). Acute and profuse bleedings may also occur in subjects on anticoagulant therapy in whom a defective haemostasis has been induced by the therapy given. Such subjects may need surgical interventions in case the anticoagulant effect has to be counteracted rapidly. Radical retropubic prostatectomy is a commonly performed procedure for subjects with localized prostate cancer. The operation is frequently complicated by significant and sometimes massive blood loss. The considerable blood loss during prostatectomy is mainly related to the complicated anatomical situation, with various densely vascularized sites that are not easily accessible for surgical haemostasis, and which may result in diffuse bleeding from a large area. Another situation that may cause problems in the case of unsatisfactory haemostasis is when subjects with a normal haemostatic mechanism are given anticoagulant therapy to prevent thromboembolic disease. Such therapy may include heparin, other forms of proteoglycans, warfarin or other forms of vitamin K-antagonists as well as aspirin and other platelet aggregation inhibitors.

In one embodiment of the invention, the bleeding is associated with haemophilia. In another embodiment, the bleeding is associated with haemophilia with aquired inhibitors. In another embodiment, the bleeding is associated with thrombocytopenia. In another embodiment, the bleeding is associated with von Willebrand's disease. In another embodiment, the bleeding is associated with severe tissue damage. In another embodiment, the bleeding is associated with severe trauma. In another embodiment, the bleeding is associated with surgery. In another embodiment, the bleeding is associated with laparoscopic surgery. In another embodiment, the bleeding is associated with haemorrhagic gastritis. In another embodiment, the bleeding is profuse uterine bleeding. In another embodiment, the bleeding is occurring in organs with a limited possibility for mechanical haemostasis. In another embodiment, the bleeding is occurring in the brain, inner ear region or eyes. In another embodiment, the bleeding is associated with the process of taking biopsies. In another embodiment, the bleeding is associated with anticoagulant therapy.

The composition according to the invention may further comprise a TFPI-inhibitor. Such a composition should preferable be administered to subjects having haemophilia A or B.

The composition according to the invention may further comprise a factor VIII. Such a composition should preferably be administered to subjects who do not have inhibitors to factor VIII.

In this context, the term “treatment” is meant to include both prevention of an expected bleeding, such as, for example, in surgery, and regulation of an already occurring bleeding, such as, for example, in haemophilia or in trauma, with the purpose of inhibiting or minimising the bleeding. Prophylactic administration of a factor VIIa and a factor XIII is thus included in the term “treatment”.

The term “subject” as used herein is intended to mean any animal, in particular mammals, such as humans, and may, where appropriate, be used interchangeably with the term “patient”.

### ABBREVIATIONS

TF tissue factor

FVII factor VII in its single-chain, unactivated form

FVIIa factor VII in its activated form

rFVIIa recombinant factor VII in its activated form

FXIII factor XIII in its zymogenic, unactivated form

FXIIIa factor XIII in its activated form

rFXIII recombinant FXIII

rFXIIIa recombinant FXIIIa

a, a₂ alpha- or a-subunits of FXIII or rFXIII

b, b₂ beta- or b-subunits of FXIII or rFXIII

FXIII-a₂ dimeric form of FXIII containing two a-subunits

FXIII-a₂b₂ tetrameric form of FXIII containing two a- and two b-subunits

FVIII factor VIII in its zymogenic, unactivated form

rFVIII recombinant FVIII

FVIIIa factor VIII in its activated form

rFVIIIa recombinant FVIIIa

TFPI tissue factor pathway inhibitor

### PREPARATION OF COMPOUNDS

### Variant FXIII

Methods for preparing recombinant factor XIII are known in the art. See, for example, Davie et al., EP 268,772; Grundmann et al., AU-A-69896/87; Bishop et al., Biochemistry 1990, 29: 1861-1869; Board et al., Thromb. Haemost. 1990, 63: 235-240; Jagadeeswaran et al., Gene 1990, 86: 279-283; and Bröker et al., FEBS Lett. 1989, 248: 105-110, which are incorporated herein by reference in their entirety. Within one embodiment, the factor XIII a₂ dimer is prepared cytoplasmically in the yeast Saccharomyces cerevisiae as disclosed in U.S. patent application Ser. No. 07/741,263, incorporated herein by reference in its entirety. The cells are harvested and lysed, and a cleared lysate is prepared. The lysate is fractionated by anion exchange chromatography at neutral to slightly alkaline pH using a column of derivatized agarose, such as DEAE Fast-Flow Sepharose™ (Pharmacia) or the like. Factor XIII is then precipitated from the column eluate by concentrating the eluate and adjusting the pH to 5.2-5.5, such as by diafiltration against ammonium succinate buffer. The precipitate is then dissolved and further purified using conventional chromatographic techniques, such as gel filtration and hydrophobic interaction chromatography.

Factor XIII variants may also be expressed as described in Lai T S, Santiago M A, Achyuthan K E, Greenberg C S (1994) Protein Expr Purif. 5:125-32.

A variant factor XIII may be produced by modifying the nucleic acid sequence encoding wild-type factor XIII either by altering the amino acid codons or by removal of some of the amino acid codons in the nucleic acid encoding the natural factor VII by known means, e.g. by site-specific mutagenesis.

### Factor VII or Variant Factor VII

Human purified factor VIIa suitable for use in the present invention is preferably made by DNA recombinant technology, e.g. as described by Hagen et al., (1986) Proc Natl Acad Sci USA 83: 2412-2416, or as described in European Patent No. 200.421 (ZymoGenetics, Inc.). Factor VIIa produced by recombinant technology may be authentic factor VIIa or a more or less modified factor VIIa provided that such a factor VIIa has substantially the same biological activity for blood coagulation as authentic factor VIIa (wild-type factor VIIa). Such a modified factor VIIa may be produced by modifying the nucleic acid sequence encoding wild-type factor VII either by altering the amino acid codons or by removal of some of the amino acid codons in the nucleic acid encoding the natural factor VII by known means, e.g. by site-specific mutagenesis.

Factor VII may also be produced by the methods described by Broze and Majerus (1980) J Biol Chem 255: 1242-1247, and Hedner and Kisiel (1983)J Clin Invest. 71: 1836-1841. These methods yield factor VII without detectable amounts of other blood coagulation factors. An even further purified factor VII preparation may be obtained by including an additional gel filtration as the final purification step. Factor VII is then converted into activated factor VIIa by known means, e.g. by several different plasma proteins, such as factor XIIa, IX a or Xa. Alternatively, as described by Bjoern et al. (Research Disclosure, 269 September 1986, pp. 564-565), factor VII may be activated by passing it through an ion-exchange chromatography column, such as Mono Q® (Pharmacia fine Chemicals) or the like.

Preparation and characterization of non-human factor XIII has been disclosed by Nakamura et al. (1975) J Biochem 78: 1247-1266. The present invention also encompasses the use of such factor XIII-variants and factor VIIa proteins within veterinary procedures.

### ADMINISTRATION AND PHARMACEUTICAL COMPOSITIONS

For treatment in connection with deliberate interventions, the factor VII and the factor XIII-variant may be administered within about 24 hours prior to performing the intervention, such for example within about 12 hours, for example within about 6 hours such as within about 3 hours, for example within 1 hour prior to performing the intervention.

For treatment in connection with deliberate interventions, the factor VII and the factor XIII-variant may also be administered during the intervention or shortly after, such as up to 48 hours, preferably up to 36 hours, such as 24 hours, preferably up to 18 hours, such as up to 12 hours, preferably up to 6 hours, such as 3 hours, e.g. up to 1 hour after the intervention.

The factor VII and the factor XIII-variant will typically be administered for as much as 1 day or more after the intervention such as 3 days or more, for example 5 days or more, such as 7 days or more thereafter. Administration is typically continued as long as an effect of the administration is observed. Administration as a coagulant can be by a variety of routes as described herein.

The dose of the factor VII ranges from about 0.05 mg to about 500 mg/day, e.g., from about 1 mg to about 200 mg/day, or, e.g., from about 2 mg/day to about 100 mg/day, preferably from about 3 mg/day to about 50 mg/day, e.g. from about 3.5 mg/day to 25 mg/day for a 70-kg subject as loading and maintenance doses. The dose will depend on the weight of the subject, the condition and the severity of the condition.

The dose of the factor XIII-variant ranges from about 0.05 mg to about 500 mg/day, e.g., from about 1 mg to about 200 mg/day, or, e.g., from about 2 mg/day to about 100 mg/day, preferably from about 3 mg/day to about 75 mg/day, such as from 4 mg/day to 50 mg/day, e.g from 5 mg/day to 30 mg/day, such as 10 to 25 mg/day for a 70-kg subject as loading and maintenance doses. The dose will depend on the weight of the subject, the condition and the severity of the condition.

The compositions and kits of the present invention are useful within human and veterinary medicine; such as, for example, in the treatment or prophylaxis of subjects suffering from bleeding episodes or coagulative disorders. For use within the present invention, the factor VIIa and factor XIII-variant are formulated, optionally with a pharmaceutically acceptable carrier. Preferably, the pharmaceutical compositions are administered parenterally, i.e., intravenously, subcutaneously, or intramuscularly, or it may be administered by continuous or pulsatile infusion.

Formulations may further include one or more diluents, emulsifiers, preservatives, buffers, excipients, etc. and may be provided in such forms as liquids, powders, emulsions, controlled release, etc. One skilled in this art may formulate the compositions of the invention an appropriate manner, and in accordance with accepted practices, such as those disclosed in Remington's Pharmaceutical Sciences, Gennaro, ed., Mack Publishing Co., Easton, Pa., 1990. The compositions for parenteral administration comprise a factor VII and a factor XIII in combination with, preferably dissolved in, a pharmaceutically acceptable carrier, preferably an aqueous carrier. A variety of aqueous carriers may be used, such as water, buffered water, 0.4% saline, 0.3% glycine and the like. The factor VII variants of the invention can also be formulated into liposome preparations for delivery or targeting to the sites of injury. Liposome preparations are generally described in, e.g., U.S. Pat. No. 4,837,028, U.S. Pat. No. 4,501,728, and U.S. Pat. No. 4,975,282.

A typical pharmaceutical composition for intravenous infusion could be made up to contain 250 ml of sterile Ringer's solution and 10 mg of a factor VIIa and/or a factor XIII-variant. Actual methods for preparing parenterally administrable compositions will be known or apparent to those skilled in the art and are described in more detail in, for example, Remington's Pharmaceutical Sciences, 18th ed., Mack Publishing Company, Easton, Pa. (1990).

In short, pharmaceutical compositions suitable for use according to the present invention is made by mixing a factor VIIa, or a factor XIII-variant, or a factor VIIa in combination with a factor XIII-variant, preferably in purified form, with suitable adjuvants and a suitable carrier or diluent. Suitable physiological acceptable carriers or diluents include sterile water and saline. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Suitable adjuvants also include calcium, proteins (e.g. albumins), or other inert peptides (e.g. glycylglycine) or amino acids (e.g. glycine, or histidine) to stabilise the purified factor VIIa and/or factor XIII-variant. Other physiological acceptable adjuvants are non-reducing sugars, polyalcohols (e.g. sorbitol, mannitol or glycerol), polysaccharides such as low molecular weight dextrins, detergents (e.g. polysorbate) and antioxidants (e.g. bisulfite and ascorbate). The adjuvants are generally present in a concentration of from 0.001 to 4% w/v. The pharmaceutical composition may also contain protease inhibitors, e.g. aprotinin or tranexamic acid, and preserving agents. Furthermore, the preparation may also contain a TFPI-inhibitor and/or factor VIII.

The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The concentration of a factor VIIa, a factor XIII-variant, or a factor VIIa in combination with a factor XIII in these formulations can vary widely, i.e., from less than about 0.5% by weight, usually at or at least about 1% by weight to as much as 15 or 20% by weight and will be selected primarily by fluid volumes, viscosities, etc., in accordance with the particular mode of administration selected.

Administration by injection or infusion, in particular injection, is preferred. Thus, the factor VIIa and the factor XIII-variant are prepared in a form suitable for intravenous administration, such as a preparation that is either a dissolved lyophilised powder or a liquid formulation containing both the factor VIIa and the factor XIII in one dosage form, or a dissolved lyophilised powder or a liquid formulation containing the factor VIIa in one dosage form and dissolved lyophilised powder or a liquid formulation containing the factor XIII-variant in another dosage form.

Local delivery of a factor VIIa and a factor XIII-variant, such as, for example, topical application may be carried out, for example, by means of a spray, perfusion, double balloon catheters, stent, incorporated into vascular grafts or stents, hydrogels used to coat balloon catheters, or other well established methods. For ambulatory subjects requiring daily maintenance levels, the factor VIIa and the factor XIII-variant may be administered by continuous infusion using e.g. a portable pump system. In any event, the pharmaceutical compositions should provide a quantity of a factor VIIa and a factor XIII-variant sufficient to effectively treat the subject.

The combination of a factor VIIa and a factor XIII-variant shows an improved effect compared to rFVIIa alone in an in vitro clot formation and stability assay (see example 5). Moreover, the data demonstrates that FXIII V34L when added to blood already containing FXIII has significant effect on clot formation and mechanical strength at a lower concentration than those required for effect of wtFXIII.

The compositions containing a factor VII and a factor XIII-variant can be administered for prophylactic and/or therapeutic treatments. In therapeutic applications, compositions are administered to a subject already suffering from a disease, as described above, in an amount sufficient to cure, alleviate or partially arrest the disease and its complications. An amount adequate to accomplish this is defined as an “effective amount” or “therapeutically effective amount”. As will be understood by the person skilled in the art, amounts effective for this purpose will depend on the severity of the disease or injury as well as the weight and general state of the subject. It must be kept in mind that the materials of the present invention may generally be employed in serious disease or injury states, that is, life threatening or potentially life threatening situations. In such cases, in view of the minimisation of extraneous substances and general lack of immunogenicity of factor VIIa and factor XIII-variants in humans, it is possible and may be felt desirable by the treating physician to administer a substantial excess of these compositions.

In prophylactic applications, compositions containing a factor VIIa and a factor XIII-variant are administered to a subject susceptible to or otherwise at risk of a disease state or injury to enhance the subject's own coagulative capability. Such an amount is defined to be a “prophylactically effective dose.”

Single or multiple administrations of the composition or compositions can be carried out with dose levels and patterns being selected by the treating physician. The compositions may be administered one or more times per day or week. An effective amount of such a pharmaceutical composition is the amount that provides a clinically significant effect against bleeding episodes. Such amounts will depend, in part, on the particular condition to be treated, age, weight, and general health of the subject, and other factors evident to those skilled in the art.

The composition or compositions may be administered before the expected bleeding; when the bleeding begins or after the bleeding has commenced. The composition or compositions may be administered as a single dose or doses. It may however also be given in multiple doses, preferably with intervals of e.g. 1, 2, 4, 6, or 12 hours, depending on the dose given and the condition of the subject. For example, in case the haemostatic effect is only achieved at doses of FXIII a-subunit exceeding the levels of free endogenous b-subunit, multiple doses given at relatively short intervals may be required to sustain a high factor XIII variant level as surplus of factor XIII variant a-subunit, not complexed with endogenous b-subunits, will be cleared relatively fast.

The composition may be in the form of a single preparation comprising both a factor VIIa and a factor XIII-variant, or a factor XIII-variant alone, in suitable concentrations. The composition may also be in the form of a kit consisting of a first unit dosage form comprising a factor VIIa and a second unit dosage form comprising a factor XIII-variant and, optionally, one or more further unit dosage forms comprising a factor VIII and/or an TFPI inhibitor. In this case, the factor VIIa and the factor XIII-variant should be administered sequentially, preferably within about 1-2 hours of each other, for example within 30 minutes of each other or, preferably, within 10 minutes or, more preferred, within 5 minutes of each other. Either of the two unit dosage forms can be administered first.

Since the present invention relates to the prevention or treatment of bleeding episodes or for coagulative treatment by treatment with a combination of active ingredients that may be administered separately, the invention also relates to combining separate pharmaceutical compositions in kit form. The kit includes at least two separate pharmaceutical compositions. The kit includes container means for containing the separate compositions such as a divided bottle or a divided foil packet. Typically the kit includes directions for the administration of the separate components. The kit form is particularly advantageous when the separate components are preferably administered in different dosage forms, are administered at different dosage intervals, or when titration of the individual components of the combination is desired by the prescribing physician.

### ASSAYS

### Test For Factor XIII Or XIII-Variant Activity

A suitable assay for testing for factor XIII transglutaminase activity and thereby selecting suitable factor XIII variants can be performed as described in the examples below. Alternatively a simple in vitro test as described, for example, in Methods of Enzymology, Vol. 45 (1976), Proteolytic Enzymes, Part B, pages 177-191 (Ed. Lorand, L.) may be used.

The present invention is further illustrated by the following examples, which, however, are not to be construed as limiting the scope of protection. The features disclosed in the foregoing description and in the following examples may, both separately and in any combination thereof, be material for realizing the invention in diverse forms thereof.

### EXAMPLES

### General Procedures

### Plasmids and DNA

All expressions plasmids are of the C-POT type, similar to those described in WO EP 171 142, which are characterized by containing the Schizosaccharomyces pombe triose phosphate isomerase gene (POT) for the purpose of plasmid selection and stabilization in S. cerevisiae.

### Yeast Strain And Transformation

S. cerevisiae strain MT663 (MATa/α pep4-3/pep4-3 HIS4/his4 tpi1::LEU2/tpi1::LEU2 Cir+) or S. cerevisiae strain ME1719 (MATa/α leu2/leu2 HIS4/HIS4 pep4-3/pep4-3 Δtpi::LEU2/Δtpi::LEU2 Δura3/Δura3 Δyps1::URA3/Δyps1::ura3 Cir+) were used as host cells for transformation. MT663 was deposited in the Deutche Sammlung von Mikroorganismen and Zellkulturen in connection with filling WO 92/11378 and was given the deposit number DSM 6278. Strain ME1719 is described in WO 98/01535. Transformation of strain MT663 and ME1719 were conducted as described in patent WO 97/22706 and WO 98/01535, respectively.

### Fermentation.

Strains with constructs based on the CIT promoter are cultivated in fed-batch fermentations using both glucose and ethanol as carbon sources. Inoculum is prepared in a batch cultivation using 6% glucose as carbon source. The main fermenter containing a peptide rich medium is inoculated by the batch culture in an amount of 12.5% on volume basis. The growth medium consists of: 50 g/L of liquid yeast extract (50% dry matter), 3.6 g/L KH₂PO₄, 2.3 g/L K₂SO₄, 1.5 g/L MgSO₄-7H₂O, 0.064 g/K FeSO₄-7H₂O, 0.016 g/L MnSO₄-H₂O, 0.011 g/L CuSO₄-5H₂O, 0.016 g/L ZnSO₄-7H₂O, 0.8 g/L citric acid, 2 g/L minositol, 0.2 g/L choline chloride, 0.2 g/L thiamine, HCl, 0.1 g/L pyridoxine, HCl, 0.2 g/L niacinamide, 1 g/L Ca-pantothenic acid, 0.005 g/L biotin, 0.05 g/L p-aminobenzoic acid, 0.66 mL/L of antifoam agent (a PEO/PPO block copolymer). Following inoculation continuous addition of a glucose solution (26 % w/w) is initiated. This fed-batch growth on glucose is continued for 42 hours and followed by 6 hours of fed-batch growth on ethanol. Basic fermentation parameters are: temperature: 28° C., pH=5.8, aeration 1-2 vvm, head space pressure 0.5 baro. pH is maintained through addition of 17% (w/w) NH₄OH.

The culture broth is harvested upon termination of the fermentation process and the biomass is isolated by centrifugation.

### Purification of FXIII/FXIII Variants

FXIII are expressed intracellular in Yeast cells in native confirmation.

The purification process consists of 4 steps.
1. Cell disruption of yeast cells.
2. Isolation of supernatant containing FXIII.
3. Purification of FXIII crude sample on Anion-Exchange Chromatography Column.
4. Further purification of FXIII for final purity on a Hydrophobic Interaction Chromatography column.
5. Concentration and buffer change on Anion Exchange Chromatography column.

Yeast cells are harvested from culture and resuspended in buffer at pH 8 and stabilizing agents (ex. Di- and tri-oler) and protease inhibitors.

The suspension is passed through a cell disrupter which breaks the cell wall by high pressure (1 -2 kBar).

Supernatant is isolated from the cell debris by filtration or centrifugation.

The supernatant containing FXIII (wt or variants) is adjusted to pH 8 and applied on an anion-exchange column, one possibility is Source30Q (Amersham Ge cat no 17-1275).

After application the column is washed with equilibration buffer at low conductivity. Increasing conductivity elutes the sample in a gradient. It is important to stabilize the protein by adding a di-ol and tri-ol (propandiol and glycerol ex.)

Pool from Anion exchanger column is adjusted to high conductivity with K-PO4 buffer. This sample is then applied on a HIC column (Phenyl og Butyl-column). The column is washed with equilibration buffer at high conductivity and elution performed by decreasing the conductivity gradually. The buffers used can be K-PO4 at diff. Conductivities.

The final pool is stabilised by adding di-ol and tri-ol.

Concentration can be done on anion-exchange column by loading high amounts of protein in low conductivity buffer. Elution is done stepwise so the pool contains FXIII in high concentration.

Elution is performed with NaCl and di-ol or tri-ol, the final pool is adjusted to PBS condition (with di-ol or tri-ol).

The product is frozen before as final soluble preparation.

The purity and yield is followed by SDS-page and by a HPLC-IEX system.

### Example 1

### Construction of a Yeast Expression_System For Human FXIII_{V34}L

DNA encoding human wtFXIII was obtained from cDNA from spleen (Stratagene) using PCR. Briefly, DNA encoding wtFXIII was amplified in three separate PCR reactions using PfuUltra Hotstart DNA polymerase (Stratagene #600392): DNA fragment 1 was amplified using oligonucleotides oMJ398 (GACCTTGTGAATTCAAAAATGTCAGAAACTTCCAG) and oMJ387 (GTTTAGAATTCACGTTCCCATC); DNA fragment 2 was amplified using oligonucleotides oMJ388 (GATGGGAACGTGAATTCTAAAC) and oMJ396 (CCTTCTTGAACTCTGCCTTCGGG); DNA fragment 3 was amplified using oligonucleotides oMJ395 (CCCGAAGGCAGAGTTCAAGAAGG) and ASA-F13-1 (TCTCAGCTTCCTCTAGATTCACATGGAAGGTCGTCTTTGAATCTG). The PCR reaction contained 10 μM of each oligonucleotide, 2.5 μl spleen cDNA, 5μl 10× PfuUltra buffer, 2.5 mM dNTP, 5 μl DMSO, 1 μl PfuUltra polymerase and 21.5 μl H₂O. After an initial incubation at 94° C. for 2 minutes, the PCR reaction was run for 35 cycles (94° C. for 30 sec., 55° C. for 30 sec., 72° C. for 1 minute) followed by a 10 minute incubation at 72° C. The resulting PCR fragments were run on agarose gels and purified using GFX-PCR Gel Band Purification Kit (Amersham Biosciences #27-9602-01). DNA fragments 2 and 3 were combined using PCR with the oligonucleotides oMJ388 and ASA-F13-1 resulting in DNA fragment 2b. DNA fragment 2b was purified as described above and combined with DNA fragment 1 using PCR with the oligonucleotides oMJ386 and ASA-F13-1, resulting in a DNA fragment encoding the complete wtFXIII sequence furnished with a unique EcoRI site at the 5′ end and a unique Xbal site at the 3′ end. This DNA fragment was cut with EcoRI and Xbal and ligated into pBluescript SK cut with the same restriction enzymes. The resulting plasmid was named pMD005.

In order to construct the V34L mutation a DNA fragment was amplified from pMD005 using the M13 forward primer in combination with oMJ343 (GTTGACGCCCCGGGGCACCAAGCCCTGAAG). The resulting PCR fragment was cut with EcoRI and Xmal and ligated to the EcoRI/Ndel vector fragment of pMD005 and the pMD005 Ndel/Xmal fragment of 171 bp. The resulting plasmid was named #2. This plasmid was digested with EcoRI/Xbal and the DNA fragment encoding FXIII_{V34L} was obtained after agarose gel electrophoresis. This fragment was ligated to the Ncol/Xbal vector fragment from pME2835, identical to the Ncol/Xbal vector fragment of pAK729.6 described in patent WO 00/04172 and the Ncol/EcoRI fragment containing the TPI1 promoter or the the Ncol/EcoRI fragment containing the CIT1 promoter from a plasmid obtained as described in patent WO0244388. The resulting plasmids were named pAW001 and pAW002 respectively The expression plasmids were propagated in E. coli, grown in the presence of ampicillin and isolated using standard techniques (Sambrook et al., 1989). The plasmid DNA was checked for insert by appropriate restriction nucleases (e.g. EcoRI, Xbal) and was shown by sequence analysis to contain the proper sequence of FXIII_{V34L}.

The plasmids pAW001 and pAW002 were transformed into S. cerevisiae strain ME1719. Yeast transformants harbouring plasmids pAW001 and pAW002 were selected by glucose utilization as carbon source on YPD (1% yeast extract, 2% peptone, 2% glucose) agar (2%) plates. One transformant from each, yAW001 and yAW002, were selected for fermentation.

### Example 2

### Construction of a Yeast Expression₁₃ System For Human FXIII_{V34L, V35T}

A FXIII variant, human FXIII V34L, V35T, with potential for faster thrombin cleavage rate was constructed by PCR. The 50 μl PCR amplifications were carried out with Expand™ High Fidelity PCR system (Roche, Switzerland) using 50-100 ng templates, 0.4-2 mM primer pair, 200 mM dNTPs and 2U of DNA polymerase. The extension reaction was initiated by pre-heating the reaction mixture to 94° C. for 30 sec followed by 20 cycles of 94° C. for 30 sec, 55° C. for 30 sec and 72° C. for 60 sec. The PCR-amplification products were evaluated by agarose gel electrophoresis and the PCR products were purified by QIAquick™ PCR purification kit (Qiagen, Germany). An 818 bp. PCR product was amplified using oligonucleotides oFISu086 (CTTGTCAAATTGAATTTTC) and oFISu087 (GTTGACGCCCCGGGGAGTCAAGCCCTGAAGCTCC) and pAW002 as template. The purified PCR product was digested with EcoRI-Xmal resulting in a 139 bp fragment that was ligated to a 1252 bp EcoRI-Ncol fragment and a 11362 bp Ncol-Xmal fragment from pAW002 to give pFE029. After verification of the nucleotide sequence, pFE029 was used for transformation of yeast strains ME1719 and MT663 resulting in intracellular expression of the variant FXIII V34L,V35T. Yeast transformants harbouring plasmid pFE029 were selected by glucose utilization as carbon source on YPD (1% yeast extract, 2% peptone, 2% glucose) agar (2%) plates. One transformant from each yeast strain, yFE029 and yFE029A respectively, was selected for fermentation.

### Example 3

### Analysis of FXIII Activation Rate By Thrombin Cleavage Assay

The FXIII a₂-subunit is activated by thrombin cleavage. Thrombin cleaves the peptide bond on the C-terminal side of Arg37 and releases FXIIIa (the activated protein) and the activation peptide (residues 1-37).

The rate of which thrombin cleaves and thus activates FXIII was analysed in a HPLC based assay basically as described in Trumbo and Maurer (2000) J Biol Chem 275: 20627-20631 and Balogh et al. 2000 Blood. 96: 2479-86 with a few modifications. The activation reaction was initiated by mixing rFXIII with human thrombin (Roche) in a buffer composed of 100 mM Tris/HCl pH 7.5, 150 mM NaCl, 5 mM CaCl₂, 0.1% PEG8000 (total volume 125 μl). The rFXIII concentration was kept constant at 7 μM (monomeric concentration) while thrombin was varied between 1-10 nM (wt), 0.3-3 nM (V34L), 0.2-1.2 nM (V34L,V35T). The reaction was carried out at 30° C. for 10 min (wt, V34L, V34L,V35T) and quenched with 25 μl 2% trifluoroacetic acid (TFA). The quenched reaction mixture was stored in glass auto sampler vials (Waters 186000234) at 4° C. until HPLC analysis. The reaction products were separated by reversed phase HPLC analysis using a 4.6×250 mm C8 column (Grace Vydac 208TP54) on a Waters Alliance HPLC system (Waters 2695 Separations Module) with a photodiode array detector (Waters 2996). Absorbance was measured at 214 nm and 280 nm. Solvent A was 0.1% TFA in H₂O and solvent B was 0.085% TFA in CH₃CN. The column was kept at 30° C. and the auto sampler was kept at 4° C. during the HPLC runs. The system was equilibrated in 15% B solvent at 1 ml/min before 100 μl of the quenched sample was injected. A linear gradient from 15%B to 50% B was run at 1 ml/min over 20 min in order to elute the activation peptide. The peak corresponding to the activation peptide was identified based on retention time and the absence of signal at 280 nm combined with a signal at 214 nm. Retention times were 10.2 min (wt), 11.6 min (V34L) and 10.4 min (V34L,V35T). The activation peptide peak was quantified using Waters Millenium software. A standard curve was generated by activating rFXIII protein fully and thus converting rFXIII entirely to rFXIIIa +activation peptide. The initial precise rFXIII concentration was known and various amounts of the solution (3.3-333 pmol) were injected on the HPLC in order to create the standard curve of signal at 214 nm versus pmol activation peptide. The rates of activation peptide production were plotted against the thrombin concentration for each rFXIII variant and the turnover number was then determined by the slope of the linear fitted curves. KaleidaGraph (Synergy software) was used for data fitting and presentation.

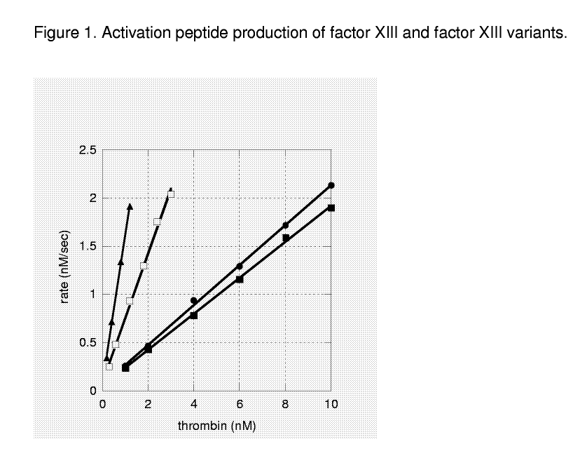

Activation peptide production plotted against thrombin concentration is shown in FIG. 1. Wild type factor XIII is shown in filled squares and circles (double determination), V34L in open squares and V34L, V35T as filled triangles. The slope of the fitted curve reflects the turnover number for thrombin when using the respective rFXIII variant as substrate.

As summarized in the table below, both FXIII V34L and FXIII V34L,V35T variants are activated faster by thrombin compared to wt. The turnover numbers of thrombin are increased 3.2 and 7.5 fold for V34L and V34L,V35T, respectively.

Turnover numbers:

| | thrombin turnover | |
|---|---|---|
| | (sec⁻¹) | ratio to wt |
| rFXIII wt* | 0.207 | 1 |
| rFXIII wt* | 0.187 | — |
| rFXIII V34L | 0.671 | 3.2 |
| rFXIII V34L, V35T | 1.56 | 7.5 |

*Double determination of wt made on two different days.
Thrombin turnover numbers measured agree within 10% of each other.

### Example 4

### Effect of FXIII on Clot Lysis

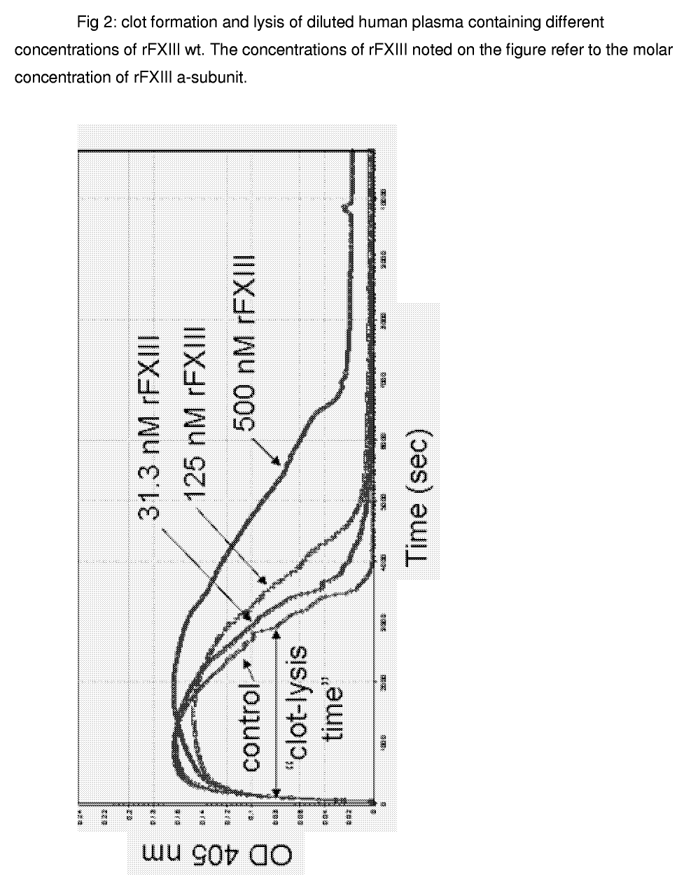

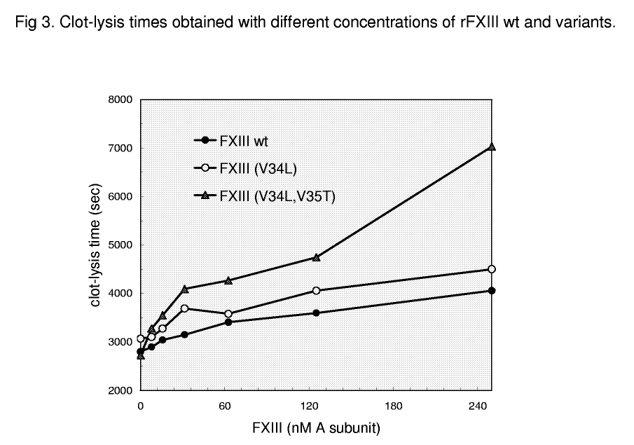

The effect of rFXIII and variants on stabilizing clots against tPA-induced lysis was analyzed in the following assay: rFXIII or rFXIII variants were diluted in 20 mM HEPES, pH 7.4, containing 150 mM NaCl, 3.5 mM KCl, and 0.1 % BSA),and 100 μl mixed with 20 μl tPA (final concentration 0.2 nM), and 20 μl normal human citrated plasma pool in microtiter plates. Clotting were initiated by adding 60 μl thrombin (final concentration 0.02 U/ml) mixed with calcium (final concentration 20 mM), and optical density (OD) at 405 nm measured continuously for 3 hrs in a SpectraMax 340 micro plate reader (Molecular Devices). FIG. 2 shows the optical density of samples containing different concentrations of rFXIII wt. When the clot is formed an increase in OD is observed. After reaching the maximal OD, the tPA-induced fibrinolysis dissolves the clot thereby decreasing the OD. rFXIII dose-dependently increased time to clot lysis demonstrating that rFXIII improves the resistance of the clot against fibrinolysis. This resistance to fibrinolysis can be quantitated by measuring the time from half-maximal OD is achieved until half-maximal OD reached during the lysis phase (the “clot-lysis time” of the control sample is noted on FIG. 2). FIG. 3 shows the clot lysis time of various concentrations of wt FXIII and rFXIII variants. At equimolar concentrations rFXIII V34L, V35T had a larger clot-stabilizing effect than rFXIII V34L, which in turn was more effective than wt FXIII in stabilizing the clot against fibrinolysis.

### Example 5

### Effect of FXIII at Thrombocytopenic Conditions in Whole Blood

The effect of rFXIII variants on clot formation and stability were evaluated by thromboelastography (TEG) at conditions mimicking thrombocytopenia. A 5000 series TEG analyzer (Haemoscope Corporation) was used for the analyses. The platelet density of citrate-stabilized normal human blood was determined on a Medonic CA 620 blood cell counter. The blood was kept at room temperature for 30 min, and then diluted with a human plasma pool to obtain a final platelet density of 10,000 platelets/μl. rFVIIa (final concentration 25 nM), wildtype rFXIII A-subunit (wtFXIII) or variants hereof (final concentration 30 or 120 nM), and combinations of rFVIIa and FXIII in a total volume of 12.5 μl were added to 20 μl 15 mM CaCl₂ in TEG sample cups. All dilutions were made in 20 mM hepes, 150 mM NaCl, pH 7.4 (HBS). A volume of 320 μl of the thrombocytopenic blood was mixed gently with 12.5 μl buffer containing human tissue factor (Innovin, Dade Behring, final dilution 1:50,000) and tPA (American Diagnostica, final concentration 1.8 nM) and immediately added to the sample cups, The clot formation was followed by continuous measurement for 120 min. All analyses were carried out in duplicate. Table 1 show values for maximal rate of thrombus formation (MTG) and maximal strength of the clot (maximal amplitude, MA). Samples containing 25 nM FVIIa and 30 nM FXIII V34L showed significantly larger MA than clots formed with 25 nM rFVIIa and 30 nM wtFXIII (p<0.05, two-tailed paired t-test), and clots formed with FVIIa and 120 nM FXIII V34L showed significantly larger MA as well as MTG than clots formed with rFVIIa and 120 nM wtFXIII (p<0.01 and p<0.05, respectively, two-tailed paired t-test). The data demonstrates that the clot stabilizing effect of FXIII V34L was significantly enhanced compared to that of wtFXIII.

TABLE 1
Thromboelastography of FVIIa, wtFXIII and FXIIIV34L
added to thrombocytopenic blood.

| | Maximal rate | | | |
|---|---|---|---|---|
| | of thrombus | | Maximal | |
| | formation (MTG) | | amplitude (MA) | |
| Sample | (mm × 100/sec) | | (mm) | |
| Control | 6.5 | (3.5-8.4) | 29.8 | (24.4-34.2) |
| FVIIa 25 nM | 8.2 | (6.8-9.3) | 29.8 | (26.2-32.1) |
| FVIIa + wtFXIII 30 nM | 8.2 | (6.6-9.4) | 30.3 | (26.0-34.2) |
| FVIIa + FXIII V34L | 8.7 | (7.3-9.7) | 32.8 | (28.9-35.0) *¹ |
| 30 nM | | | | |
| FVIIa + wtFXIII 120 nM | 9.1 | (8.1-9.7) | 33.6 | (31.4-35.1) |
| FVIIa + FXIII V34L | 10.0 | (8.9-11.3) *¹ | 37.7 | (34.1-41.0) **¹ |
| 120 nM | | | | |

¹ Significant different (* with p < 0.05 and ** with p < 0.01) from samples with same amount of wtFXIII, two-tailed paired t-test
Concentrations are nM of rFXIII a-subunit. The values show the mean values and range of data for six experiments.

### Example 6

### Effect of FXIII at FXIII-Deficiency

The effect of FXIII variants in FXIII-deficient plasma supplemented with normal platelets was evaluated by thromboelastography as described in example 5. Platelet-rich plasma was prepared from citrate-stabilized normal human blood by 10 min centrifugation at 10 min centrifugation at 200 x g. Platelets were isolated by gel filtration on a Speharose CL6B column equilibrated in 15 mM Hepes, pH 7.4, 138 mM NaCl, 5 mM CaCl₂, 2.7 mM KCl, 1 mM MgCl₂, 5.5 mM dextrose, and 1 mg/ml BSA, and gently pelleted by 4 min centrifugation at 312 x g. The platelets were reconstituted with FXII I-deficient plasma (George King) to a final platelet density of 150,000/μl in the assay, and thromboelastography carried out as described in example 5. The MTG and MA values for the FXIII variants are shown in table 2. When equimolar concentrations of wt FXIII, FXIII V34L, and FXIII V34L, V35T were compared, FXIII V34L increased MTG and MA more than wt FXIII did, and FXIII V34L, V35L increased MTG and MA more than FXIII V34L did. This demonstrates that the FXIII variants are more effective than wt rFXIII in clot formation and stability.

TABLE 2
Thromboelastography of wtFXIII, FXIII V34L and
FXIII V34L, V35T added to FXIII deficient plasma
supplemented with normal platelets.

| | Maximal rate | |
|---|---|---|
| | of thrombus | Maximal |
| | formation (MTG) | amplitude (MA) |
| Sample | (mm × 100/sec) | (mm) |
| Control | 6.6 ± 0.7 | 18.9 ± 2.2 |
| 60 nM wt FXIII | 18.6 ± 0.6 | 57.5 ± 3.2 |
| 60 nM FXIII V34L | 19.3 ± 0.1 | 59.1 ± 2.1 |
| 60 nM FXIII V34L, V35T | 19.6 ± 0.0 | 60.5 ± 1.5 |
| 120 nM wt FXIII | 19.1 ± 0.2 | 59.5 ± 1.9 |
| 120 nM FXIII V34L | 19.6 ± 0.2 | 62.5 ± 2.5 |
| 120 nM FXIII V34L, V35T | 20.4 ± 0.2 | 65.6 ± 1.3 |
| 240 nM wt FXIII | 19.0 ± 0.5 | 60.7 ± 3.1 |
| 240 nM FXIII V34L | 21.1 ± 0.4 | 68.2 ± 1.0 |
| 240 nM FXIII V34L, V35T | 22.0 ± 0.1 | 70.6 ± 1.3 |

The values show the mean and standard deviations for three experiments. The concentrations are the molar concentration of FXIII-a subunit.

1-7. (canceled)

8. A pharmaceutical composition comprising a variant factor XIII comprising at least one modification of the amino acid sequence in the region comprising position 28-41 of the activation peptide such that the rate of activation of said variant by thrombin is faster than for wild type FXIII.

9. (canceled)

10. A pharmaceutical composition according to claim 8, wherein the modification or modifications is selected from the group consisting of T28D, V29F, E30L, L31A, Q32E, V34(G, L), V35(M,K,Q,R,E,H,L,T,N,S,A), P36(L,V), G38(S,A), V39(F,Y,W,R,M), N40(R,K,W,H,Q,A,S) and L41V.

11. A pharmaceutical composition according to claim 8, wherein the residues in position 34-40 has been replaced by an amino acid sequence selected from the group of VVPRSFR, VLPRSFR, VTPRSFR, LLPRSFR, LTPRSFR, VVPRSYR, VLPRSYR, VTPRSYR, LLPRSYR, LTPRSYR or LTPRGVN.

12. A pharmaceutical composition according to claim 8 further comprising a factor VIIa or a variant factor VIIa.

13. A kit-of-parts comprising a factor XIII variant comprising at least one modification of the amino acid sequence in the region comprising position 28-41 of the activation peptide such that wherein the rate of activation of said variant by thrombin is faster than for wild type FXIII in a first container and a factor VIIa or variant factor VIIa in a second container.

14-17. (canceled)

18. A factor XIII variant, wherein at least one amino acid in a region comprising position 28-41 of the activation peptide has been modified, the modification or modifications is being selected from the group consisting of T28D, V29F, E30L, L31A, Q32E, V34(G), V35(M,K,Q,R,E,H,T,N,S,A), P36(L,V), G38(S,A), V39(F,Y,W,R,M), N40(R,K,W,H,Q,A,S) and L41V.

19. A factor XIII variant according to claim 18, wherein the residues in position 34-40 been replaced by the amino acid sequence VVPRSFR or VLPRSFR or VTPRSFR or LLPRSFR or LTPRSFR or VVPRSYR or VLPRSYR or VTPRSYR or LLPRSYR or LTPRSYR or LTPRGVN.

20. A pharmaceutical composition according to claim 10 further comprising factor VIIa or a variant factor VIIa.

21. A pharmaceutical composition according to claim 11 further comprising factor VIIa or a variant factor VIIa.

22. A method for enhancing fibrin clot formation in a subject comprising administering to the subject an effective amount of a composition according to claim 8.

23. The method of claim 22, wherein the composition is a composition according to claim 10.

24. The method of claim 22, wherein the composition is a composition according to claim 11.

25. The method of claim 22, wherein the method further comprises administering an effective amount of a factor VIIa or a variant factor VIIa to the subject.

26. The method of claim 23, wherein the method further comprises administering an effective amount of a factor VIIa or a variant factor VIIa to the subject.

27. The method of claim 24, wherein the method further comprises administering an effective amount of a factor VIIa or a variant factor VIIa to the subject.

28. The method of claim 22, wherein the subject is a human.

29. The method of claim 23, wherein the subject is a human.

30. The method of claim 24, wherein the subject is a human.

31. The method of claim 26, wherein the factor VIIa is recombinant human factor VIIa and the subject is a human.

32. The method of claim 27, wherein the factor VIIa is recombinant human factor VIIa and the subject is a human.

33. A kit according to claim 13, wherein the at least one modification in the factor XIII variant is selected from the group consisting of T28D, V29F, E30L, L31A, Q32E, V34(G, L), V35(M,K,Q,R,E,H,L,T,N,S,A), P36(L,V), G38(S,A), V39(F,Y,W,R,M), N40(R,K,W,H,Q,A,S) and L41V.

34. A kit according to claim 13, wherein the residues in position 34-40 of the factor XIII variant has been replaced by an amino acid sequence selected from the group consisting of VVPRSFR, VLPRSFR, VTPRSFR, LLPRSFR, LTPRSFR, VVPRSYR, VLPRSYR, VTPRSYR, LLPRSYR, LTPRSYR, and LTPRGVN.
